(19) United States
(12) Patent Application Publication
(10) Pub. No.: US 2006/0122452 A1
(43) Pub. Date: Jun. 8, 2006

(54) Brachytherapy catheter needle

(75) Inventor: Eric van't Hooft, Brasschaat (BE)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Isodose Controll Intellectual Property B.V.i.o., Veenendaal (NL)

(21) Appl. No.: 11/286,551

(22) Filed: Nov. 23, 2005

(30) Foreign Application Priority Data
Nov. 23, 2004 (NL) 1027562

(51) Int. Cl. A61N 5/00

(52) U.S. Cl. 600/3

(57) ABSTRACT

A brachytherapy catheter needle formed as an elongate pointed leading part terminating in a coupling piece for fixation of the brachytherapy catheter behind and in line with the leading part. Such a structure is by nature stiff. The brachytherapy catheter needle can be pulled entirely through the tissue, so that the brachytherapy catheter can be cut at the exit side. This method can be performed faster and more effectively than with the conventional brachytherapy catheter needles. The brachytherapy catheter needle according to the invention is preferably relatively short and solid and can be inserted into the tissue in front of the brachytherapy catheter tube. This provides the advantage that the needle can be made of relatively stiff design, enabling a better straight guidance.

### CROSS-REFERENCE TO RELATED APPLICATIONS

This patent application claims priority to Netherlands Patent Application NL 1027562 filed Nov. 23, 2004. The contents of which are expressly incorporated by reference herein in their entirety including any references therein.

### AREA OF THE INVENTION

This invention, relates to brachytherapy catheter needles of a type for inserting a brachytherapy catheter into a part of the body.

### BACKGROUND OF THE INVENTION

In some therapeutic treatments, in particular patient radiation therapy for the treatment of tumors, it is necessary to provide a plurality of brachytherapy catheters in the tissue. For instance in the treatment of breast tumors in women, it is conventional usage to clamp the breast between two templates and, with the breast in a compressed condition, placing the brachytherapy catheters, via openings provided, in the templates. Subsequently, radioactive sources are positioned in the brachytherapy catheters to irradiate a tumor present in the breast without unduly burdening the surrounding tissue. For the patient, a treatment with such a device, whereby the breast is flattened, is very unpleasant, often necessitating total anesthesia. A further drawback of the breast being flattened is that the target volume to be treated is enlarged, which necessitates additional needles to enable sufficiently homogeneous irradiation of the target site. In the case of a radiation treatment taking several days, a patient must tolerate the hard flat templates protractedly to avoid a risk of dosimetric errors due to the distance between the needles not having been, kept equal.

Further, use is made of conventional brachytherapy catheter needles. Such needles are cylinder-shaped hollow structures which, after placement in the tissue, function as guide tubes for a thin guide connected to the brachytherapy catheter. After the thin guide has been wholly arranged in the open needle, needle and brachytherapy catheter are jointly pulled through the tissue, so that the brachytherapy catheter ends up in place. Then the brachytherapy catheter is cut off and a counter stopper is slipped onto it. The cut portion is then measured off, and used as coupling tube to connect a guide tube. The hollow brachytherapy catheter needles are not very strong and moreover rather expensive.

### BRIEF SUMMARY OF THE INVENTION

An object of the invention is to provide a device whereby a more acceptable treatment with a better dose distribution can, be offered, which is simpler to use.

This object is achieved by a device according to the features of the claimed invention. In particular, the invention comprises a brachytherapy catheter needle, which is formed as an elongate pointed leading part terminating in a coupling piece for fixation of the brachytherapy catheter behind and in line with the leading part. Such a structure is by nature much stiffer, so that the use of a solid template for controlling the direction is necessary to a much lesser extent. In addition, during breast radiation treatment, the brachytherapy catheter needle can be pulled entirely through the tissue, so that the brachytherapy catheter can be cut through at the exit side. This method can be performed faster and more effectively than with the conventional brachytherapy catheter needles. The brachytherapy catheter needle according to the invention is preferably relatively short and solid and can be inserted into the tissue in front of the brachytherapy catheter tube. This provides the advantage that the needle can be made of relatively stiff design, enabling a better straight guidance.

### BRIEF DESCRIPTION OF THE DRAWINGS

While the claims set forth the features of the present invention with particularity, the invention, together with its objects and advantages, may be best understood from the following detailed description taken in conjunction with the accompanying drawing of which:

FIG. 1 shows a conventional brachytherapy catheter; and

FIG. 2 shows a schematic representation of a brachytherapy catheter according to the invention.

### DETAILED DESCRIPTION OF THE DRAWINGS

In the Figures, the same or corresponding parts are designated by the same reference numerals.

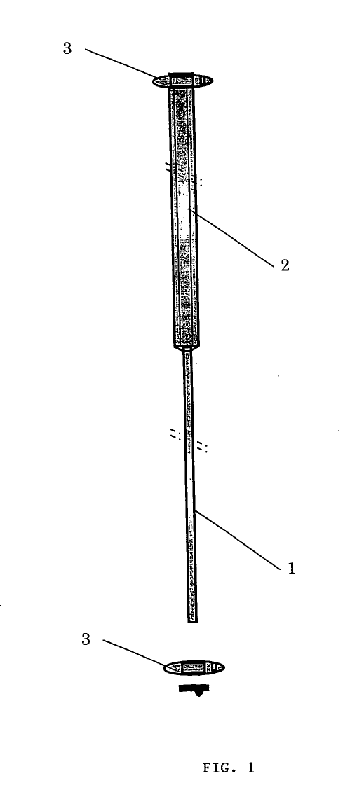

In FIG. 1 a conventional brachytherapy catheter is shown. By means of a hollow needle (not shown) a thin stiff guide wire 1 is introduced into the body, over which a brachytherapy catheter 2 can be pushed into the body. In a treatment in which the needle is pulled entirely through the body, the needle and needle guide are pulled out of the body at the exit side. On the brachytherapy catheter 2, a stop element 3 is optionally provided to prevent the tube sliding into the body too far.

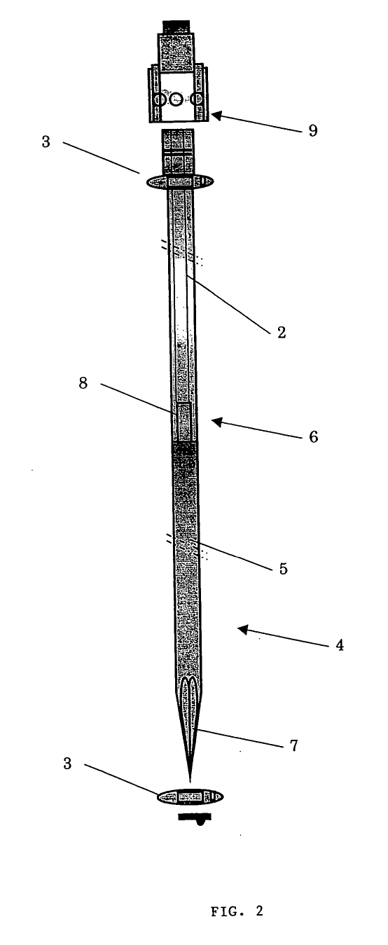

FIG. 2 shows the preferred embodiment of the invention. Here, the brachytherapy catheter needle 4 is formed as an elongate leading part 5 which terminates in a coupling piece 6 for fixation of the brachytherapy catheter 2 behind and in alignment with the leading part 5. In use, this brachytherapy catheter needle 4, in particular in the case of breast radiation treatments, can simply be inserted through the tissue, thereby passing the brachytherapy catheter 2 through the tissue. When the brachytherapy catheter 2 has been pulled through, the needle can be cut off from the brachytherapy catheter 2 at the exit side, eliminating the need to remove the needle via the brachytherapy catheter 2 at the entry side.

By virtue of such a brachytherapy catheter needle 4, a great saving of time can be accomplished when implanting brachytherapy catheters for irradiating tumors, for instance in the female breast. Also, this needle 4 can be made of considerably cheaper design than the conventional brachytherapy catheter needles. The needle is preferably of solid design, so that during insertion it will barely bend (if at all), thereby achieving a high mutual parallelism between brachytherapy catheters. The solid elongate leading part 5 can be provided with a very sharp tip 7. Provided at the side remote from the tip 7 is the coupling piece 6. In particular, the coupling piece 6 is formed by a tailing part protruding from the needle body, for insertion in a brachytherapy Catheter 2, the coupling piece 6 being smaller in diameter than the diameter of the needle 4. In this example the coupling piece 6 forms a squeeze fitting 8 with brachytherapy Catheter 2. However, the coupling piece 6 can also be formed by a bracket which encloses the brachytherapy Catheter 2. Accordingly, the brachytherapy Catheter 2 is coupled behind and in line with the brachytherapy catheter needle 4 and a smooth transition between needle 4 and brachytherapy catheter 2 is achieved. In particular, the needle diameter is larger than the diameter of the brachytherapy catheter 2.

FIG. 2 further shows schematically that the bracytherapy catheter 2 is coupled with a coupling element 9 of a radiation dosing device (not shown). What can be achieved through stop elements 3 which are slipped over the brachytherapy Catheter 2 or are fixedly fitted thereon is that the brachytherapy Catheter 2 is fixed in the tissue and axial shifts are avoided by stopping the brachytherapy catheter at the front and/or the back.

The invention has been elucidated on the basis of the preferred embodiment. It is to be understood here that other embodiments falling within the scope of the invention are also possible. For instance, the brachytherapy catheter can be straight or curved and used with or without templates.

What is claimed is:

1. A brachytherapy catheter needle for inserting a brachytherapy catheter into a part of the body, characterized in that:
the brachytherapy catheter needle is formed as an elongate pointed leading part which terminates in a coupling piece for fixation of the brachytherapy catheter behind, and in line with, the leading part.

2. A brachytherapy catheter needle according to claim 1, characterized in that the leading part is of solid design.

3. A brachytherapy catheter needle according to claim 1, wherein a needle diameter is larger or equal to a brachytherapy catheter diameter.

4. A brachytherapy catheter needle according to claim 1, wherein the coupling piece is smaller in diameter than the needle diameter for insertion in a brachytherapy catheter, for providing a smooth transition between needle and brachytherapy catheter.

5. A brachytherapy catheter needle according to claim 1, characterized in that the coupling piece comprises a squeeze fitting.

6. A brachytherapy catheter needle according to claim 1, characterized in that the brachytherapy catheter needle comprises a stopper for stopping the brachytherapy catheter.

7. A brachytherapy catheter needle according to claim 6 wherein the stopper stops the brachytherapy catheter at the front.

8. A brachytherapy catheter needle according to claim 6 wherein the stopper stops the brachytherapy catheter at the back.

9. A brachytherapy catheter needle according to claim 6 wherein the stopper stops the brachytherapy catheter at both the front and back.

10. A brachytherapy catheter needle according to claim 1, characterized in that the brachytherapy catheter needle is fixedly connected with the brachytherapy catheter.

11. A brachytherapy catheter needle according to claim 10, characterized in that the brachytherapy catheter comprises a fixed stop element.
